(19) United States
(12) Patent Application Publication
(10) Pub. No.: US 2006/0104943 A1
(43) Pub. Date: May 18, 2006

(54) Vaccine comprising il-13 and an adjuvant

(76) Inventors: Claire Ashman, Hertfordshire (GB); Jonathan Ellis, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/526,151

(22) PCT Filed: Aug. 28, 2003
(86) PCT No.: PCT/GB03/03721

§ 371 Date: Feb. 28, 2005

(30) Foreign Application Priority Data
Aug. 30, 2002 (GB) 0220211.7
Feb. 28, 2003 (GB) 0304672.9

(51) Int. Cl. A61K 48/00; A61K 38/20

(52) U.S. Cl. 424/85.2; 514/44

(57) ABSTRACT

The present invention relates to IL-13 vaccines and their use in the treatment of diseases that are treatable with neutralization of IL-13 such as COPD, asthema and atopic disorders such as hayfever, contact allergies and atopic dermatitis. The vaccines of the present invention comprise an IL-13 immunogen and an adjuvant composition which is a combination of saponin and an immunostimulatory oligonucleotide comprising at least one unmethylated dinucleotide. The invention further relates to pharmaceutical compositions comprising such immunogens and their use in medicine and to methods for their protection.

The present invention relates to IL-13 vaccines and their use in the treatment of diseases that are treatable with neutralisation of IL-13, such as COPD, asthma and atopic disorders such as hayfever, contact allergies and atopic dermatitis. The vaccines of the present invention comprise an IL-13 immunogen and an adjuvant composition which is a combination of a saponin and an immunostimulatory oligonucleotide comprising at least one unmethylated dinucleotide. The invention further relates to pharmaceutical compositions comprising such immunogens and their use in medicine and to methods for their production.

### BACKGROUND TO THE INVENTION

COPD is an umbrella term to describe diseases of the respiratory tract, which shows similar symptoms to asthma and is treated with the same drugs. COPD is characterised by a chronic, progressive and largely irreversible airflow obstruction. The contribution of the individual to the course of the disease is unknown, but smoking cigarettes is thought to cause 90% of the cases. Symptoms include coughing, chronic bronchitis, breathlessness and respiratory injections. Ultimately the disease will lead to severe disability and death.

Asthma is a chronic lung disease, caused by inflammation of the lower airways and is characterised by recurrent breathing problems. Airways of patients are sensitive and swollen or inflamed to some degree all the time, even when there are no symptoms. Inflammation results in narrowing of the airways and reduces the flow of air in and out of the lungs, making breathing difficult and leading to wheezing, chest tightness and coughing. Asthma is triggered by super-sensitivity towards allergens (e.g. dust mites, pollens, moulds), irritants (e.g. smoke, fumes, strong odours), respiratory infections, exercise and dry weather. The triggers irritate the airways and the lining of the airways swell to become even more inflamed, mucus then clogs up the airways and the muscles around the airways tighten up until breathing becomes difficult and stressful and asthma symptoms appear.

Atopic disorders refers to a group of diseases that are hereditary and often occur together, including asthma, allergies such as hay fever, and atopic dermatitis. Atopic dermatitis is a chronic disease that affects the skin. In atopic dermatitis, the skin becomes extremely itchy and inflamed, causing redness, swelling, cracking, weeping, crusting, and scaling. Atopic dermatitis most often affects infants and young children, but it can continue into adulthood or first show up later in life. In most cases, there are periods of time when the disease is worse, called exacerbations or flares, followed by periods when the skin improves or clears up entirely, called remissions. Many children with atopic dermatitis will experience a permanent remission of the disease when they get older, although their skin often remains dry and easily irritated. Environmental factors can bring on symptoms of atopic dermatitis at any time in the lives of individuals who have inherited the atopic disease trait. Atopic dermatitis is often referred to as “eczema,” which is a general term for the many types of dermatitis. Atopic dermatitis is the most common of the many types of eczema Several have very similar symptoms.

The way the skin is affected by atopic dermatitis can be changed by patterns of scratching and resulting skin infections. Some people with the disease develop red, scaling skin where the immune system in the skin is becoming very activated. Others develop thick and leathery skin as a result of constant scratching and rubbing. This condition is called lichenification. Still others develop papules, or small raised bumps, on their skin. When the papules are scratched, they may open (excoriations) and become crusty and infected.

Many factors or conditions can make symptoms of atopic dermatitis worse, further triggering the already overactive immune system in the skin, aggravating the itch-scratch cycle, and increasing damage to the skin. These exacerbating factors can be broken down into two main categories: irritants (such as wool or synthetic fibers, rough or poorly fitting clothing, soaps and detergents, some perfumes and cosmetics, chlorine, mineral oil, some solvents, dust or sand) and allergens (such as pollen, dog or cat dander, and dust mite allergens). Emotional factors and some infections can also influence atopic dermatitis.

If a flare of atopic dermatitis does occur, several methods can be used to treat the symptoms. Corticosteroids as topical creams are the most frequently used treatment, although systemic administration is also used in some severe cases. Sometimes over-the-counter preparations are used, but in many cases the doctor will prescribe a stronger corticosteroid cream or ointment. An example of a commonly prescribed corticosteroid is prednisone. Side effects of repeated or long-term use of topical corticosteroids can include thinning of the skin, infections, growth suppression (in children), and stretch marks on the skin. Antibiotics to treat skin infections may be applied directly to the skin in an ointment, but are usually more effective when taken by mouth. Phototherapy (treatment with light) that uses ultraviolet A or B light waves, or both together, can be an effective treatment for mild to moderate dermatitis in older children (over 12 years old) and adults. In adults, immunosuppressive drugs, such as cyclosporine, are also used to treat severe cases of atopic dermatitis that have failed to respond to any other forms of therapy. The side effects of cyclosporine can include high blood pressure, nausea, vomiting, kidney problems, headaches, tingling or numbness, and a possible increased risk of cancer and infections.

Because of the unmet medical need therefor and the side affects of existing therapies there is a need for alternative treatments for atopic diseases in general, and in particular for treatments for asthma and atopic dermatitis.

IL-13 is a Th2-type cytokine that is closely related to IL-4. A number of recent papers have defined the role for IL-13 in driving pathology in the ovalbumin model of allergenic asthma (Wills-Karp et al, 1998, Science 282:2258-2261; Grunig et al, 1998, Science 282:2261-2263). In this work, mice previously sensitised to ovalbumin were injected with a soluble IL-13 receptor which binds and neutralises IL-13. Airway hyper-responsiveness to acetylcholine challenge was reduced in the treated group. Histological analysis revealed that treated mice had reversed the goblet-cell metaplasia seen in controls. In complementary experiments, lung IL-13 levels were raised by over-expression in a transgenic mouse or by installation of protein into the trachea in wild-type mice. In both settings, airway hyper-responsiveness, eosinophil invasion and increased mucus production were seen (Zhu et al, 1999, J. Clin. Invest. 103:779-788).

The sequence of the mature form of human IL-13 is provided in SEQ ID No. 1 and is shown in FIG. 1.

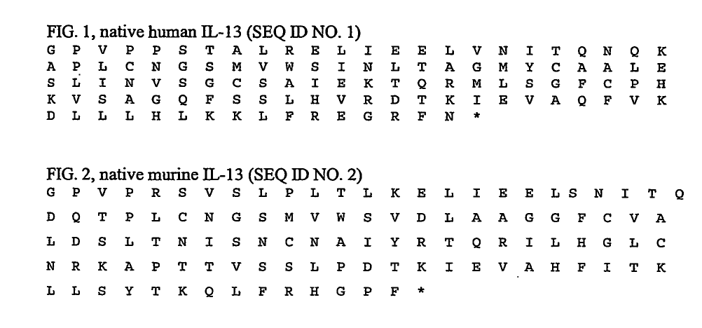

The sequence of the mature form of murine IL-13 is provided in SEQ ID No. 2 and is shown in FIG. 2.

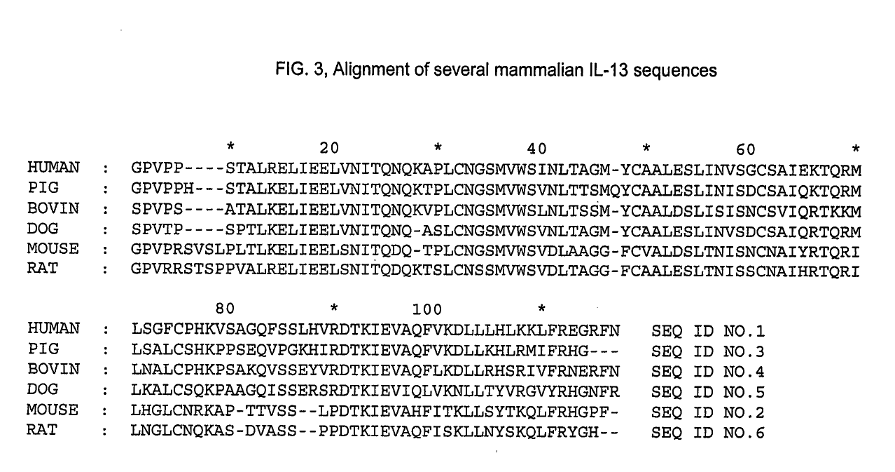

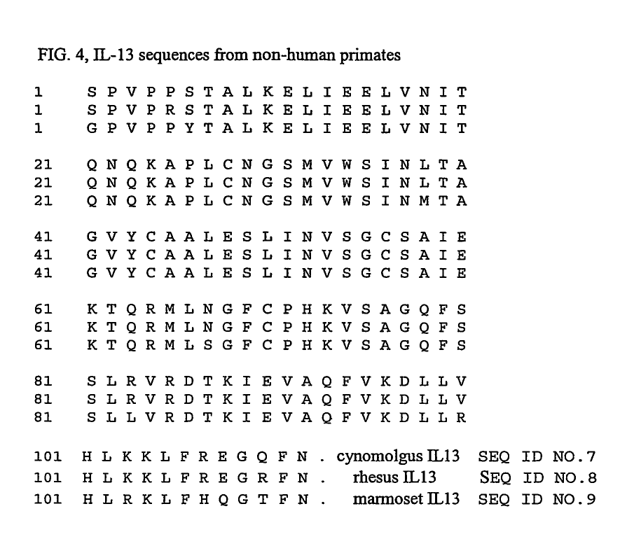

Sequences for IL-13 from several mammalian species and non-human primates are shown in FIG. 3 and FIG. 4 (SEQ ID NOs 3 to 9)

As a result of the various problems associated with the production, administration and tolerance of monoclonal antibodies there is an increased focus on methods of instructing the patient's own immune system to generate endogenous antibodies of the appropriate specificity by means of vaccination. However, mammals do not generally have high-titre antibodies against self-proteins present in serum, as the immune system contains homeostatic mechanisms to prevent their formation. The importance of these “tolerance” mechanisms is illustrated by diseases like myasthenia gravis, in which auto-antibodies directed to the nicotinic acetylcholine receptor of skeletal muscle cause weakness and fatigue (Drachman, 1994, N Engl J Med 330:1797-1810).

A number of techniques have been designed with the aim of breaking “tolerance” to self antigen. One technique involves chemically cross-linking the self-protein (or peptides derived from it) to a highly immunogenic carrier protein, such as keyhole limpet haemocyanin (“Antibodies: A laboratory manual” Harlow, E and Lane D. 1988. Cold Spring Harbor Press).

A variant on the carrier protein technique involves the construction of a gene encoding a fusion protein comprising both carrier protein (for example hepatitis B core protein) and self-protein (The core antigen of hepatitis B virus as a carrier for immunogenic peptides”, Biological Chemistry. 380(3):277-83, 1999). The fusion gene may be administered directly as part of a nucleic acid vaccine. Alternatively, it may be expressed in a suitable host cell in vitro, the gene product purified and then delivered as a conventional vaccine, with or without an adjuvant.

Another approach has been described by Dalum and colleagues wherein a single class II MHC-restricted epitope is inserted into the target molecule. They demonstrated the use of this method to induce antibodies to ubiquitin (Dalum et al, 1996, J Immunol 157:4796-4804; Dalum et al, 1997, Mol Immunol 34:1113-1120) and the cytokine TNF (Dalum et al, 1999, Nature Biotech 17:666-669). As a result, all T cell help must arise either from this single epitope or from junctional sequences. Such an approach is also described in EP 0 752 886 B1, WO 95/05849, and WO 00/65058.

Treatment therapies, some including vaccination, for the neutralisation of several cytokines are known. WO 00/65058 describes a method of down regulating the function of the cytokine IL-5, and its use in the treatment of asthma. In this study, the IL-5 sequence was modified by a number of techniques to render it immunogenic, amongst which there is described an IL-5 immunogen supplemented with foreign T-cell epitopes, whilst maintaining the IL-5 B cell epitopes. WO 01/62287 discloses IL-113, amongst a long list of potential antigens, for use in allergy or asthma vaccines. WO 00/06937 discloses cytokine derivatives that are functionally inactivated for use as vaccine antigens. Chimaeric IL-13 immunogens are disclosed in the co-pending patent application WO 02/070711.

Current treatments of chronic asthma and COPD require frequent and regular administration of therapeutic drugs, which in the case of short acting beta2 agonists can be required several times per day. There is a need for improved treatment methods which do not require such frequent administrations, and for improved vaccines for raising neutralising anti-IL-13 immune responses.

### SUMMARY OF THE INVENTION

The present invention provides novel vaccine formulations for the treatment of asthma or COPD comprising an immunogen that is capable of generating an immune response in a vaccinee against self IL-13 and an adjuvant compositions comprising a combination of a saponin and an immunostimulatory oligonucleotide comprising at least one unmethylated dinucleotide.

Preferably the vaccine formulations comprise modified “self” IL-13 immunogens, wherein the IL-13 immunogen is modified to include foreign T-cell helper epitopes. The vaccine is preferably for use in human therapy, and in this composition the IL-13 sequence is a human sequence or other sequence that is capable of generating an immune response that recognises human IL-13; and the T-cell helper epitopes are “foreign” with respect to human self-proteins. Preferably the T-helper epitopes are also foreign with respect to other IL-13 sequences from other species. However, animal pharmaceutical products are not excluded, for example canine or other veterinary species pharmaceutical products can be made in an analogous fashion to that described for human vaccines above.

Use of the vaccines in medicine is provided by the present invention. The vaccines of the present invention, or immunogens and adjuvant combinations described herein, are used in the manufacture of medicaments for the treatment of asthma or COPD, and use in novel methods of treatment of asthma or COPD. Also provided by the present invention are methods of manufacturing vaccines of the present invention.

In all aspects of the present invention there is an immunogen that is capable of generating an immune response in a vaccinee against self IL-13. In the case of a human asthma vaccine the immunogen is any immunogen that is capable, when formulated in vaccines of the present invention, of generating an anti-human IL-13 immune response. Preferably the immune response is an antibody response, and most preferably an IL-13 neutralising antibody response that neutralises the biological effects of IL-13 in asthma disease.

The compositions of the present invention comprise an IL-13 immunogen, which may comprise an additional element for providing T-cell help, and an adjuvant combination comprising a saponin and an immunostimulatory oligonucleotide comprising at least one unmethylated dinucleotide.

Immunogen

The vaccines of the present invention comprise an immunogen which raises an immune response against IL-13, and may comprise a polypeptide sequence corresponding to IL-13 (the IL-13 element) which may further comprise an additional element to provide T-cell help.

IL-13 Element

The IL-13 element, in its broadest form, is any sequence that is capable of driving an immune response that recognises and neutralises the biological effects of IL-13. Preferably, the IL-13 is human IL-13.

In this context of the present invention the entire IL-13 sequences may be used, or functional equivalent fragments thereof. Accordingly, references in this text to IL-13 sequences may encompass the entire sequence or fragments or truncates thereof.

The IL-13 element may comprise the native IL-13 sequence or a mutated form thereof. Accordingly, the IL-13 sequence may be, for example, native human IL-13 or fragment thereof.

As the vaccines of the present invention are to raise an immune response against a self-protein, the immunogens of the present invention preferably comprise human IL-13, or immunogenic fragment thereof, which has been rendered immunogenic in a “self” situation (that is to say for use in vaccination of a human with a human protein sequence as the immunogen).

In one such embodiment of the present invention, the immunogens comprise a chimaeric IL-13 sequence that comprises substitution mutations to swap one or more of the human sequence amino acids with the equivalent amino acids found in the same positions within the sequence of IL-13 from another mammalian species. In the context of a human vaccine immunogen, the object of the chimaeric sequences is to maximise the amino acid sequence diversity between the immunogen and human native IL-13, whilst keeping maximal shape and conformational homology between the two compositions. The chimaeric immunogen achieves this by substituting amino acids found in regions predicted to be masked from the surface. Most preferably the amino acids are substituted with amino acids that are found in equivalent positions within an IL-13 sequence from another mammalian species. In this way, sequence diversity is achieved with minimal alteration to the overall shape/configuration of the immunogen.

In one aspect of the present invention, the human IL-13 immunogen comprises substitution mutations in areas that are associated with alpha helical regions, which substitutions involve swapping the human amino acid with the amino acid that appears in the same position within the IL-13 sequence of a different mammalian species.

Most preferably, there are substitution mutations in a plurality of sites within the IL-13 sequence, wherein at least two or more of the mutation sites comprise a substitution involving amino acids taken from different non-human mammalian species, more preferably the substitutions involve amino acids taken from 3 or more different non-human mammalian species, and most preferably the substitutions involve amino acids taken from 4 or more different non-human mammalian species.

Preferably, the substitutions in the human IL-13 sequence do not occur in at least six of the areas of high interspecies conservation: 3PVP, 12ELIEEL (SEQ ID NO. 58), 19NITQ (SEQ ID NO. 59), 28LCN, 32SMVWS (SEQ ID NO. 60), 50SL, 60AI, 64TQ, 87DTKIEVA (SEQ ID NO. 61), 99LL, 106LF.

The preferred IL-13 element of the vaccines of the present invention are human chimaeric IL-13 sequences which have a similar conformational shape to native human IL-13 whilst having sufficient amino acid sequence diversity to enhance its immunogenicity when administered to a human, characterised in that the chimaeric IL-13 immunogen has the sequence of human IL-13 comprising:

(a) substitution mutations in at least two of the following alpha helical regions: PSTALRLIEELVNIT (SEQ ID NO. 24), MYCAALESLI (SEQ ID NO. 25), KTQRMLSGF (SEQ ID NO. 26) or AQFVKDLLLHLKKLFRE (SEQ ID NO. 27),

(b) comprises in unmutated form at least six of the following regions of high inter-species conservation 3PVP, 12ELIEEL (SEQ ID NO. 58), 19NITQ (SEQ ID NO. 59), 28LCN, 32SMVWS (SEQ ID NO. 60), 50SL, 60AI, 64TQ, 87DTKIEVA (SEQ ID NO. 61), 99LL, 106LF, and

(c) optionally comprises a mutation in any of the remaining amino acids, wherein any substitution performed in steps a, b or c is a structurally conservative substitution.

The numerical prefix to the amino acids listed, refers to the positional number of the amino acid sequence in the mature form of human IL-13, wherein the first residue “G” is assigned the number 2.

In the context of step (a) of the above chimaeric IL-13 element, preferably at least two, more preferably at least three and most preferably all four alpha helical regions comprise at least one substitution mutation. In the context of step (b) preferably at least 7, more preferably at least 8, more preferably at least 9, more preferably at least 10, and most preferably all 11 of the regions are unmutated.

Preferably greater than 50% of these substitutions or mutations in the above chimaeric IL-13 element, comprise amino acids taken from equivalent positions within the IL-13 sequence of a non-human. More preferably more than 60, or 70, or 80 percent of the substitutions comprise amino acids taken from equivalent positions within the IL-13 sequence of a non-human mammal. Most preferably, each substitution or mutation comprise amino acids taken from equivalent positions within the IL-13 sequence of a non-human mammal.

Again in the context of the chimaeric human IL-13 element, preferably greater than 50% of these substitutions or mutations occur in regions of human IL-13 which are predicted to be alpha helical in configuration. More preferably more than 60, or 70, or 80 percent of the substitutions or mutations occur in regions of human IL-13 which are predicted to be alpha helical in configuration. Most preferably, each substitution or mutation occurs in regions of human IL-13 which are predicted to be alpha helical in configuration.

Again in the context of the chimaeric human IL-13 elements, preferably the human IL-13 sequence comprises between 2 and 20 substitutions, more preferably between 6 and 15 substitutions and most preferably 13 substitutions in total.

In the case of a human IL-13 vaccine, the IL 13 immunogen could be based on an orthologous IL-13 sequence (such as the murine IL-13 sequence) wherein the murine B-cell epitopes (surface exposed regions) are substituted for the equivalent human sequences. In this embodiment the murine “backbone” will provide foreign T-cell epitopes, in addition to the supplemental promiscuous T-cell epitopes (such as P2 or P30) which are added either at the termini or within the chimaera sequence.

A preferred chimaeric human IL-13 immunogen for use in the vaccines of the present invention, comprises the sequence of human IL-13, wherein the amino acid sequences comprises conservative substitutions, or substitutions characteristic of amino acids present at equivalent positions within the IL-13 sequence of a non-human species, present in at least six of the following 13 positions 8T, 11R, 18V, 49E, 62K, 66M, 69G, 84H, 97K, 101L, 105K, 109E, 111R. Most preferably such a chimaeric human IL-13 immunogen comprises at least 6, and preferrably all, of the following substitutions:

| Position | Substitution | Species |
|---|---|---|
| 8 | T->S | Synthetic |
| 11 | R->K | pig, cow, dog, mouse, gerbil, |
| | | cyno, rhesus, marmoset. |
| 18 | V->A | Synthetic |
| 49 | E->D | cow, mouse, gerbil. |
| 62 | K->R | cow, dog, mouse, rat. |
| 66 | M->I | Mouse, gerbil, rat. |
| 69 | G->A | Cow, pig, dog |
| 84 | H->R | Dog, rhesus, cyno |
| 97 | K->T | Mouse |
| 101 | L->V | Cyno, rhesus |
| 105 | K->R | Synthetic |
| 109 | E->Q | Marmoset |
| 111 | R->T | Marmoset |

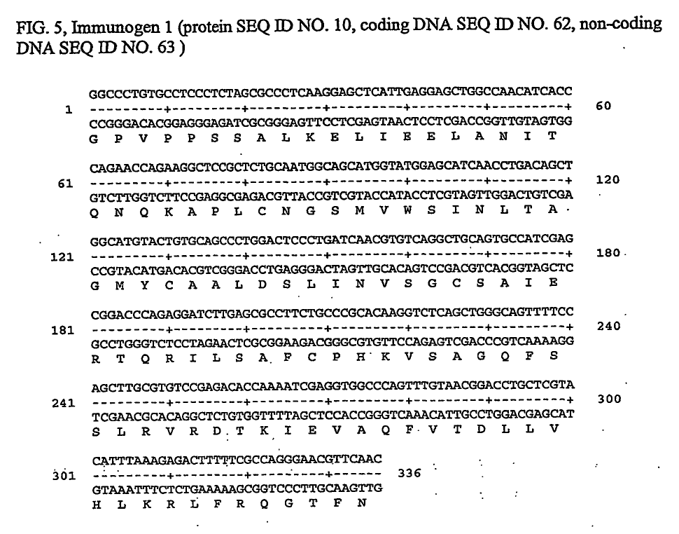

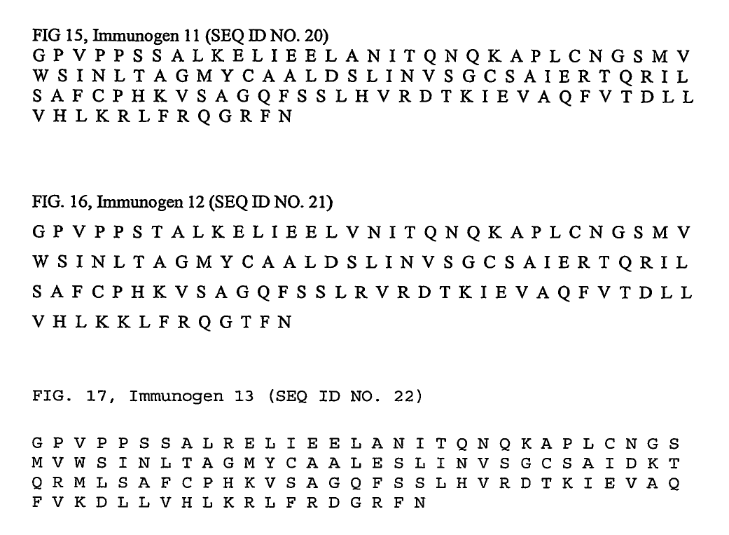

The chimaeric IL-13 that comprises each of these listed substitutions is a preferred IL-13 immunogen (Immunogen 1, SEQ ID NO. 10) and is shown in FIG. 5. Other highly preferred IL-13 immunogen are Immunogen 11 (SEQ ID NO. 20, see FIG. 15), Immunogen 12 (SEQ ID NO. 21, see FIG. 16) and Immunogen 13 (SEQ ID NO. 22, see FIG. 17).

The IL-13 element may also optionally further comprise a mutation that abolishes the biological activity of the immunogen. The following substitutions can be used to inactivate human IL13 bioactivity: E 12 to I, S, or Y; E12 to K; R 65 to D; S 68 to D; R 108 to D.

In certain aspects of the present invention immunogenic fragments of the native IL-13 sequence may be used, for example in the presentation of immunogenic peptides in Hepatitis B core particles or in the context of chimaeric immunogens described above. In these contexts immunogenic fragments of the human IL-13 sequences preferably contain the B-cell epitopes in the human IL-13 sequence, and preferably at least one or more of the following short sequences:

| | |
|---|---|
| GPVPPSTA | (SEQ ID NO. 28) |
| ITQNQKAPLCNGSMVWSINLTAGM | (SEQ ID NO. 29) |
| INVSGCS | (SEQ ID NO. 30) |
| FCPHKVSAGQFSSLHVRDT | (SEQ ID NO. 31) |
| LHLKKLFREGRFN | (SEQ ID NO. 32) |

The polypeptide of the invention may be further modified by mutation, for example substitution, insertion or deletion of amino-acids in order to add desirable properties (such as the addition of a sequence tag that facilitates purification or increase immunogenicity) or remove undesirable properties (such as an unwanted agonistic activity at a receptor) or trans-membrane domains. In particular the present invention specifically contemplates fusion partners that ease purification such as poly histidine tags or GST expression partners that enhance expression. A preferred tag or expression partner is immunoglobulin FC of human IgG1 fused to the C-terminus of the IL-13 molecule.

Other mutations, outside of those regions that are to be left unmutated due to their high level of conservation between species, may occur in the IL-13 sequence. Preferably such mutations are conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. These are preferred conservative substitutions.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

Element to Provide T-Cell Help.

In one aspect of the present invention, the IL-13 immunogen may further comprise an additional element to provide T-cell help.

Accordingly the immunogens for use in the vaccines of the present invention may comprise modified human IL-13 immunogens, wherein the human IL-13 sequence is modified to include foreign T-cell helper epitopes. The T-cell helper epitopes are preferably “foreign” with respect to human proteins, and also preferably foreign with respect to any IL-13 sequences from non-human mammals.

Preferably the T-cell helper epitopes are small and are added to the IL-13 sequence by an addition or substitution event within or at the terminal ends of the IL-13 sequence by synthetic, recombinant or molecular biological means. Alternatively the T-cell helper epitopes may be added via chemical coupling of the IL-13 polypeptide to a carrier protein comprising the T-cell helper epitopes. The IL-13 sequences, or functionally equivalent fragments thereof, may also be associated with the T-cell helper epitopes in a fusion protein, wherein the two are recombinantly manufactured together, for example a Hepatitis B core protein incorporating IL-13 sequences.

In the aspects of the present invention where small T-cell helper epitopes are used, a “foreign T-cell helper epitope” or “T-cell epitope” is a peptide which is able to bind to an MHC II molecule and stimulates T-cells in an animal species. Preferred foreign T-cell epitopes are promiscuous epitopes, ie. epitopes that bind multiple different MHC class II molecules in an animal species or population (Panina-Bordignon et al, Eur. J. Immunol. 1989, 19:2237-2242; Reece et al, J. Immunol. 1993, 151:6175-6184; WO 95/07707).

In order for the immunogens of the present invention to be clinically effective in a complex outbred human population, it may be advantageous to include several foreign T-cell epitopes. Promiscuous epitopes may also be another way of achieving this same effect, including naturally occurring human T-cell epitopes such as those from tetanus toxoid (e.g. the P2 and P30 epitopes, diphtheria toxoid, influenza virus haemagluttinin (HA), and P. falciparum CS antigen. The most preferred T-cell epitopes for use in the present invention are P2 and P30 from tetanus toxoid A number of promiscuous T-cell epitopes have been described in the literature, including: WO 98/23635; Southwood et al., 1998, J. Immunol., 160: 3363-3373; Sinigaglia et al., 1988, Nature, 336: 778-780; Rammensee et al., 1995, Immunogenetics, 41: 4, 178-228; Chicz et al., 1993, J. Exp. Med., 178:27-47; Hammer et al., 1993, Cell 74:197-203; and Falk et al., 1994, Immunogenetics, 39: 230-242. The promiscuous T-cell epitope can also be an artificial sequence such as “PADRE” (WO 95/07707).

The heterologous T-cell epitope is preferably selected from the group of epitopes that will bind to a number of individuals expressing more than one MHC II molecules in humans. For example, epitopes that are specifically contemplated are P2 and P30 epitopes from tetanus toxoid, Panina-Bordignon Eur. J. Immunol 19 (12), 2237 (1989). In a preferred embodiment the heterologous T-cell epitope is P2 or P30 from Tetanus toxin.

The P2 epitope has the sequence QYIKANSKFIGITE (SEQ ID NO. 33) and corresponds to amino acids 830-843 of the Tetanus toxin.

The P30 epitope (residues 947-967 of Tetanus Toxin) has the sequence FNNFTVSFWLRVPKVSASHLE (SEQ ID NO. 34). The FNNFTV sequence may optionally be deleted. Other universal T epitopes can be derived from the circumsporozoite protein from Plasmodium falciparum—in particular the region 378-398 having the sequence DIEKKIAKMEKASSVFNVVNS (SEQ ID NO. 35) (Alexander J, (1994) Immunity 1 (9), p 751-761).

Another epitope is derived from Measles virus fusion protein at residue 288-302 having the sequence LSEIKGVIVHRLEGV (SEQ ID NO. 36) (Partidos CD, 1990, J. Gen. Virol 71(9) 2099-2105).

Yet another epitope is derived from hepatitis B virus surface antigen, in particular amino acids, having the sequence FFLLTRILTIPQSLD (SEQ ID NO. 37).

Another set of epitopes is derived from diphteria toxin. Four of these peptides (amino acids 271-290, 321-340, 331-350, 351-370) map within the T domain of fragment B of the toxin, and the remaining 2 map in the R domain (411430, 431450):

| | |
|---|---|
| PVFAGANYAAWAVNVAQVI | (SEQ ID NO. 38) |
| VHHNTEEIVAQSIALSSLMV | (SEQ ID NO. 39) |
| QSIALSSLMVAQAIPLVGEL | (SEQ ID NO. 40) |
| VDIGFAAYNFVESII NLFQV | (SEQ ID NO. 41) |
| QGESGKDIKITAENTPLPIA | (SEQ ID NO. 42) |
| GVLLPTIPGKLDVNKSKTHI | (SEQ ID NO. 43) |

(Raju R., Navaneetham D., Okita D., Diethelm-Okita B., McCormick D., Conti-Fine B. M. (1995) Eur. J. Immunol. 25: 3207-14.)

A particularly preferred element to provide T-cell help, is a fusion partner called “CPC” (clyta-P2-clyta) which is disclosed in PCT/EP03/06096.

Most preferably the foreign T-cell helper epitopes are “foreign” in that they are not tolerated by the host immune system, and also in that they are not sequences that are derived or selected from any IL-13, sequence from another species (non-vaccinee).

In the aspect of the present invention where native self IL-13 is coupled to a T-helper epitope bearing immunogenic carrier, the conjugation can be carried out in a manner well known in the art. Thus, for example, for direct covalent coupling it is possible to utilise a carbodiimide, glutaraldehyde or (N-[γ-maleimidobutyryloxy] succinimide ester, utilising common commercially available heterobifunctional linkers such as CDAP and SPDP (using manufacturers instructions). After the coupling reaction, the immunogen can easily be isolated and purified by means of a dialysis method, a gel filtration method, a fractionation method etc.

The types of carriers used in the immunogens of the present invention will be readily known to the man skilled in the art. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT), or recombinant fragments thereof (for example, Domain 1 of Fragment C of TT, or the translocation domain of DT), or the purified protein derivative of tuberculin (PPD). Alternatively the IL-13 may be directly conjugated to liposome carriers, which may additionally comprise immunogens capable of providing T-cell help. Preferably the ratio of IL-13 to carrier molecules is in the order of 1:1 to 20:1, and preferably each carrier should carry between 3-15 IL-13 molecules.

In an embodiment of the invention a preferred carrier is Protein D from Haemophilus influenzae (EP 0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D ⅓^{rd} (comprising the N-terminal 100-110 amino acids of protein D (GB 9717953.5)).

Another preferred method of presenting the IL-13, or immunogenic fragments thereof, is in the context of a recombinant fusion molecule. For example, EP 0 421 635 B describes the use of chimaeric hepadnavirus core antigen particles to present foreign peptide sequences in a virus-like particle. As such, immunogens of the present invention may comprise IL-13 presented in chimaeric particles consisting of hepatitis B core antigen. Additionally, the recombinant fusion proteins may comprise IL-13 and a carrier protein, such as NS1 of the influenza virus. For any recombinantly expressed protein which forms part of the present invention, the nucleic acid which encodes said immunogen also forms an aspect of the present invention.

Preferred Immunogens for Use in Vaccines of the Present Invention

In the sections above, preferred definitions of the IL-13 element and, if present, the element to provide T-cell help have been described. For certain preferred compositions intended to be incorporated within vaccines of the present invention, it is intended that this document discloses each individual preferred element from the IL-13 element section in combination with each individual preferred element from the element to provide T-cell help section. Particularly preferred are combinations of immunogens 1, 11, 12 or 13, and a carrier protein or promiscuous T-cell helper epitope. Preferred carrier protein or promiscuous T-cell helper epitopes include Protein D, CPC, P2 or P30.

Specifically disclosed preferred combinations of elements to form preferred immunogens are listed herebelow.

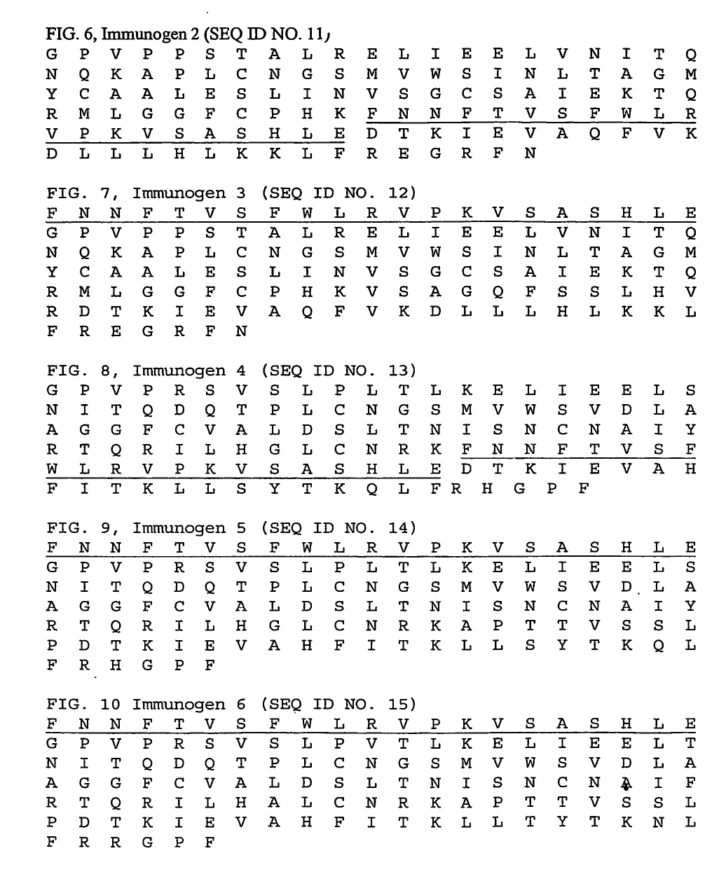

When the IL-13 element is native human IL-13, and the element that provides T-cell help is a promiscuous T-cell epitope, preferred examples include: Immunogen 2 (see FIG. 6, SEQ ID NO. 11), which comprises human IL-13 with P30 inserted (underlined) into the protein (substituted for the looped region between alpha helices C and D of human IL13).

Immunogen 3 (FIG. 7, SEQ ID NO. 12) is a Human IL 13 immunogen with N-terminal P30.

Immunogen 4 (FIG. 8, SEQ ID NO. 13) is a murine IL-13 with p30 inserted into the protein (substituted for the looped region between alpha helices C and D of mouse IL13) this is an example of a mouse version of an IL13 autovaccine. The p30 region is underlined.

Immunogen 5 (FIG. 9, SEQ ID NO. 14) is a murine IL13 with p30 at the N-terminus. This is an example of a mouse version of an IL13 autovaccine. The p30 region is underlined and is positioned at the N-terminus of the mature mouse IL13 protein sequence.

Specific examples where the IL-13 element is provided as a chimaeric IL-13 immunogen include:

Immunogen 6 (FIG. 10, SEQ ID NO. 15). This is an example of a mouse version of this form of the vaccine, where there is “human backbone” sequence grafted to murine B-cell surface exposed epitopes, with P30 added at the N-terminus.

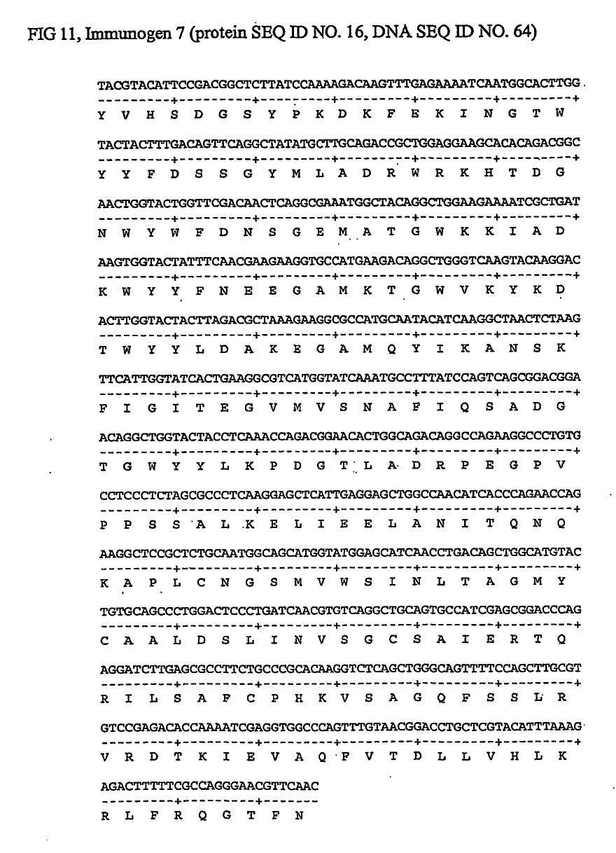

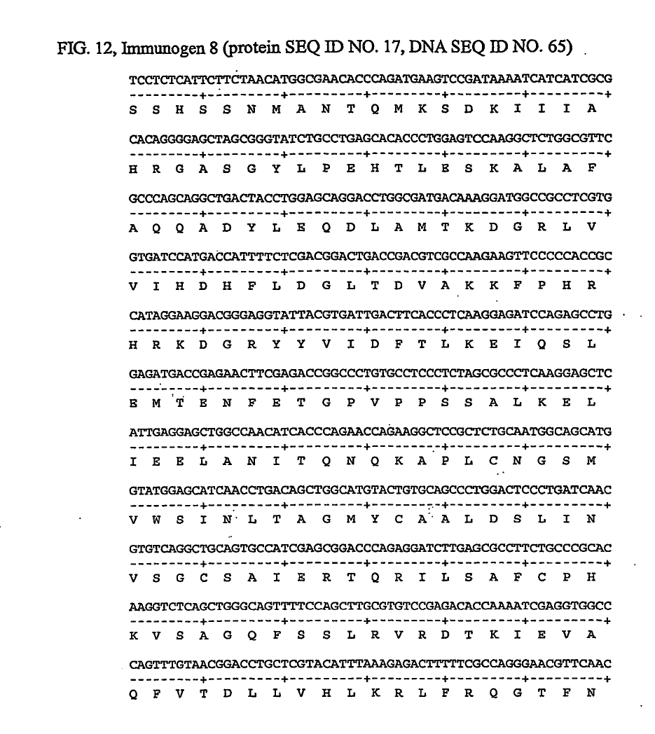

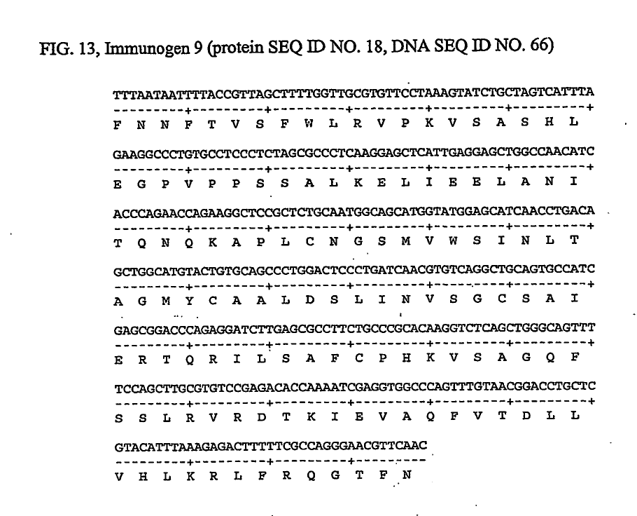

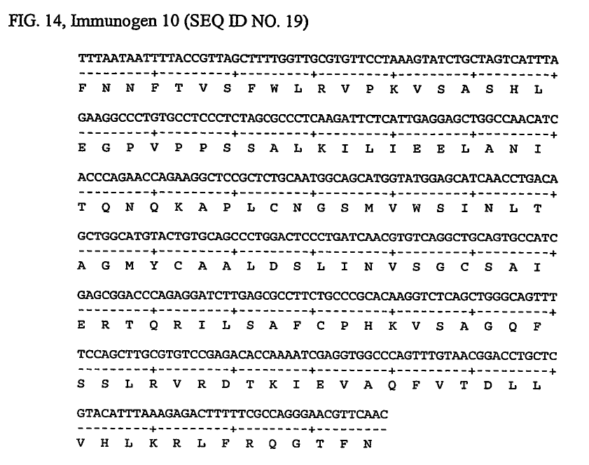

Other preferred immunogens are based on a human chimaeric IL-13 “Immunogen 1” (SEQ ID NO. 10). For example, Immunogen 1 is preferably N-terminally fused to the carrier “CPC” to form Immunogen 7 (SEQ ID NO. 16, see FIG. 11), or N-terminally fused to protein D (the protein D fusion region corresponds to amino acids S20 to T127 inclusive, of H. influenzae protein D sequence (nb, the DNA sequence encoding the protein D is codon optimised) for Immunogen 8 (SEQ ID NO. 17, see FIG. 12); or N-terminally fused to P30 to give Immunogen 9 (SEQ ID NO.18, see FIG. 13). Immunogen 9 preferably further comprises the E121 mutation to abrogate any IL-13 biological activity, to give Immunogen 10 (SEQ ID NO. 19, see FIG. 14).

The protein and DNA sequences shown for Immunogens 1 to 10 are shown without the amino acid or DNA sequence for the signal sequence required to drive secretion of the product from the cell. Preferably, therefore, the sequences further are further provided with a signal sequence. In the context of DNA vaccines it is specifically preferred that the signal sequence is a non-human derived sequence that comprises a T-cell epitope, to further provide T-cell help. None of the disclosed preferred sequences have a stop codon as it may be useful to express them fused to other molecules eg immunoglobulin Fe, 6His to facilitate production or purification.

The numbering system used herein conforms with normal practice in the field of IL-13, in that the G in “GPVPP” is referred to as residue 2, and the remaining amino acids are numbered accordingly.

In one aspect of the present invention there is provided a method for the manufacture of a human chimaeric IL-13 vaccine comprising the following steps:

(a) taking the sequence of human IL-13 and performing at least one substitution mutation in at least two of the following alpha helical regions: PSTALRELIEELVNIT (SEQ ID NO. 24), MYCAALESLI (SEQ ID NO. 25), KTQRMLSGF (SEQ ID NO. 26) or AQFVKDLLLIKLFE (SEQ ID NO. 27),

(b) preserving at least six of the following regions of high inter-species conservation 3PVP, 12ELIEEL (SEQ ID NO. 58), 19NITQ (SEQ ID NO. 59), 28LCN, 32SMVWS (SEQ ID NO. 60), 50SL, 60AI, 64TQ, 87DTKIEVA (SEQ ID NO. 61), 99LL, 106LF,

(c) optionally mutating any of the remaining amino acids,

(d) attaching a source of T-cell epitopes that are foreign with respect to any human self epitope and also foreign with respect to any mammalian IL-13 sequence, to form an IL-13 immunogen, and

(e) combining the IL-13 immunogen with an adjuvant composition comprising a saponin and an immunostimulatory oligonucleotide comprising at least one unmethylated CG dinucleotide,

characterised in that any substitution performed in steps a, b or c is a structurally conservative substitution.

In the context of step (a) preferably at least two, more preferably at least three and most preferably all four alpha helical regions comprise at least one substitution mutation. In the context of step (b) preferably at least 7, more preferably at least 8, more preferably at least 9, more preferably at least 10, and most preferably all 11 of the regions are unmutated

In all of this method, preferably greater than 50% of these substitutions or mutations comprise amino acids taken from equivalent positions within the IL-13 sequence of a non-human. More preferably more than 60, or 70, or 80 percent of the substitutions comprise amino acids taken from equivalent positions within the IL-13 sequence of a non-human mammal. Most preferably, each substitution or mutation comprise amino acids taken from equivalent positions within the IL-13 sequence of a non-human mammal.

Again in the context of the method for the manufacture of a human chimaeric IL-13 vaccine, preferably greater than 50% of these substitutions or mutations occur in regions of human IL-13 which are predicted to be alpha helical in configuration. More preferably more than 60, or 70, or 80 percent of the substitutions or mutations occur in regions of human IL-13 which are predicted to be alpha helical in configuration. Most preferably, each substitution or mutation occurs in regions of human IL-13 which are predicted to be alpha helical in configuration.

Again in the context of the method for the manufacture of a human chimaeric IL-13 vaccine, preferably the immunogen comprises between 2 and 20 substitutions, more preferably between 6 and 15 substitutions, and most preferably 13 substitutions.

Most preferably, in all of these above methods there are substitution mutations in a plurality of sites within the IL-13 sequence, wherein at least two or more of the mutation sites comprise a substitution involving amino acids taken from different non-human mammalian species, more preferably the substitutions involve amino acids taken from 3 or more different non-human mammalian species, and most preferably the substitutions involve amino acids taken from 4 or more different non-human mammalian species.

The successful design of a polypeptide according to the present invention can be verified for example by administering the resulting polypeptide in a self-context in an appropriate vaccination regime, and observing that antibodies capable of binding the protein are induced. This binding may be assessed through use of ELISA techniques employing recombinant or purified native protein, or through bioassays examining the effect of the protein on a sensitive cell or tissue. A particularly favoured assessment is to observe a phenomenon causally related to activity of the protein in the intact host, and to determine whether the presence of antibodies induced by the methods of the invention modulate that phenomenon. Thus a protein of the present invention will be able to raise antibodies to the native antigen in the species from which the native protein is derived.

The most successful of designs will be able to be used in an experiment, such as that described in Example 2 herein, and induce anti-IL-13 neutralising immune responses that exceed ED100 in at least 50% of the vaccinated individuals.

Vaccine Formulations

The immunogens as described above form vaccines of the present invention when they are formulated with adjuvants or adjuvant comprising a combination of a saponin and an immunostimulatory oligonucleotide comprising at least one unmethylated dinucleotide.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising Quil A or fractions thereof, have been used in the manufacture of vaccines (Morein, B., EP 0 109 942 B1; WO 96/11711; WO 96/33739). The saponins QS21 and QS17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

The adjuvant combinations further comprise an immunostimulatory oligonucleotide comprising an unmethylated CG dinucleotide, such as disclosed in (WO96102555). Typical immunostimulatory oligonucleotides will be between 8-100 bases in length and comprises the general formula X₁ CpGX₂ where X₁ and X₂ are nucleotide bases, and the C and G are unmethylated.

The preferred oligonucleotides for use in vaccines of the present invention preferably contain two or more dinucleotide CpG motifs preferably separated by at least three, more preferably at least six or more nucleotides. The oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages. e.g. mixed phosphorothioate/phophodiesters. Other internucleotide bonds which stabilise the oligonucleotide may be used. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

(SEQ ID NO. 44)

OLIGO 1:
TCC ATG ACG TTC CTG ACG TT (CpG 1826)

(SEQ ID NO. 45)

OLIGO 2:
TCT CCC AGC GTG CGC CAT (CpG 1758)

(SEQ ID NO. 46)

OLIGO 3:
ACC GAT GAC GTC GCC GGT GAC GGC ACC ACG

(SEQ ID NO. 47)

OLIGO 4:
TCG TCG TTT TGT CGT TTT GTC GTT (CpG 2006)

(SEQ ID NO. 48)

OLIGO 5:
TCC ATG ACG TTC CTG ATG CT (CpG 1668)

Alternative CpG oligonucleotides may comprise the preferred sequences above in that they have inconsequential deletions or additions thereto.

The CpG oligonucleotides utilised in the present invention may be synthesized by any method known in the art (eg EP 468520). Conveniently, such oligonucleotides may be synthesized utilising an automated synthesizer.

Preferably the adjuvant contains a combination of CpG and saponin as described in WO 00/62800, the entire contents of which are fully incorporated herein by reference. Such adjuvant compositions are also described in WO 00/09159. The most preferred adjuvant combinations of this subgroup comprise QS21 and OLIGO 4. Most preferably the saponin, preferably QS21, is associated with cholesterol containing liposomes, and the, immunostimulatory oligonucleotide, preferably OLIGO 4, is in aqueous solution. Alternatively, the QS21 and immunostimulatory oligonucleotide is presented in an oil in water emulsion, wherein the oil droplets comprise squalene and alpha-tocopherol and a stabilising detergent; the oil droplets optionally further comprising cholesterol (WO 99/12565).

Most preferred adjuvants comprise a mixture of small unilamellar dioleoyl phosphatidyl choline liposomes comprising cholesterol and QS21 at a cholesterol:QS21 ratio of at least 1:1 w/w and preferably with excess cholesterol; and the immunostimulatory oligonucleotide in aqueous suspension or associated with the liposome.

Another preferred adjuvant comprises an oil in water emulsion comprising an aqueous phase and an oil phase, wherein the oil phase comprises oil droplets of squalene and alpha-tocopherol and a stabilising detergent; optionally further comprising cholesterol; and admixed into the aqueous phase, QS21 and an immunostimulatory oligonucleotide.

The present invention also includes pharmaceutical or vaccine compositions, which comprise a therapeutically effective amount of vaccines of the present invention, optionally in combination with a pharmaceutically acceptable carrier, preferably in combination with a pharmaceutically acceptable excipient such as phosphate buffered saline (PBS), saline, dextrose, water, glycerol, ethanol, liposomes or combinations thereof.

Methods of Treatment

The present invention provides novel treatments for atopic diseases, comprising a vaccine that is capable of generating an immune response in a vaccinee against IL-13. Most notably the present invention provides a method of treating an individual suffering from or being susceptible to COPD, asthma or atopic dermatitis, comprising administering to that individual a vaccine according to the present invention, and thereby raising in that individual a serum neutralising anti-IL-13 immune response and thereby ameliorating or abrogating the symptoms of COPD, asthma or atopic dermatitis.

Also provided by the present invention is the use of the vaccines of the present invention in the manufacture of a medicament for the treatment asthma. Also provided is a method of treatment of asthma comprising the administration to an individual in need thereof of a pharmaceutical composition or vaccine as described herein.

Preferably the pharmaceutical composition is a vaccine that raises an immune response against IL-13. The immune response raised is preferably an antibody response, most preferably an IL-13 neutralising antibody response.

The methods of treatment of the present invention provide a method of treatment of asthma comprising one or more of the following clinical effects:

1. A reduction in airway hyper-responsiveness (AHR)

2. A reduction in mucus hyper-secretion and goblet cell metaplasia

3. A reduction in sub-epithelial fibrosis of the airways

4. A reduction in eosinophil levels

5. A reduction in the requirement for the use of inhaled corticosteroids (ICS) would also be a feature of successfull treatment using an IL13 autovaccine.

The compositions of the present invention may be used for both prophylaxis and therapy. The present invention provides a polypeptide or a polynucleotide according to the invention for use in medicine. The invention further provides the use of a polypeptide or a polynucleotide of the invention in the manufacture of a medicament for the treatment of allergies, respiratory ailments such as asthma and COPD, helminth-infection related disorders, fibrosis or cirrhosis of the liver.

The present invention also provides a method of vaccinating which comprises administering an effective amount of a vaccine composition of the invention to a patient and provoking an immune response to the vaccine composition.

The present invention also provides vaccine compositions as described herein for use in vaccination of a mammal against IL-13 mediated disorders such as allergies, respiratory ailments, helminth-infection related disorders, fibrosis and cirrhosis of the liver. A vaccine composition capable of directing a neutralising response to IL-13 would therefore constitute a useful therapeutic for the treatment of asthma, particularly allergic asthma, in humans. It would also have application in the treatment of certain helminth infection-related disorders (Brombacher, 2000 Bioessays 22:646-656) and diseases where IL-13 production is implicated in fibrosis (Chiaramonte et al, 1999, J Clin Inv 104:777-785), such as chronic obstructive pulmonary disease (COPD) and cirrhosis of the liver.

The methods of treatment of the present invention provide a method of treatment of atopic dermatitis comprising one or more of the following clinical effects:

1. A reduction in skin irritation

2. A reduction in itching and scratching

3. A reduction in the requirement for conventional treatment.

4. if applicable a reduction in the requirement for the use of topical corticosteroids.

An ideal IL13 autovaccine could potentially make ICS steroid treatment redundant, although a reduction in the ‘frequency of use’ or ‘dose required’ of ICS is also envisaged as a valuable outcome.

Administration of the vaccines of the present invention may take the form of one or more individual doses, for example in a “prime-boost” therapeutic vaccination regime. In certain cases the “prime” vaccination may be via particle mediated DNA delivery of a polynucleotide according to the present invention, preferably incorporated into a plasmid-derived vector and the “boost” by administration of a recombinant viral vector comprising the same polynucleotide sequence, or boosting with the protein in adjuvant. Conversely the priming may be with the viral vector or with a protein formulation typically a protein formulated in adjuvant and the boost with a DNA vaccine of the present invention.

The present invention provides methods of generating an anti self IL-13 antibody response in a host by the administration of vaccines of the present invention.

The vaccine compositions of the invention may be administered in a variety of manners for example via the mucosal, such as oral and nasal; pulmonary, intramuscular, subcutaneous or intradermal routes. Where the antigen is to be administered as a protein based vaccine, the vaccine will typically be formulated with an adjuvant and may be lyophilised and resuspended in water for injection prior to use. Such compositions may be administered to an individual as an injectable composition, for example as a sterile aqueous dispersion, preferably isotonic. Typically such compositions will be administered intra muscularly, but other routes of administration are possible. One technique for intradermally administration involves particle bombardment (which is also known as ‘gene gun’ technology and is described in U.S. Pat. No. 5,371,015). Proteins may be formulated with sugars to form small particles and are accelerated at speeds sufficient to enable them to penetrate a surface of a recipient (e.g. skin), for example by means of discharge under high pressure from a projecting device.

The amount of vaccine composition which is delivered will vary significantly, depending upon the species and weight of mammal being immunised, the nature of the disease state being treated/protected against, the vaccination protocol adopted (i.e. single administration versus repeated doses), the route of administration and the potency and dose of the adjuvant compound chosen. Based upon these variables, a medical or veterinary practitioner will readily be able to determine the appropriate dosage level but it may be, for example, when the vaccine is a nucleic acid that the dose will be 0.5-5 μg/kg of the nucleic acid constructs or composition containing them. In particular, the dose will vary depending on the route of administration. For example, when using intradermal administration on gold beads, the total dosage will preferably between 1 μg-10 ng, particularly preferably, the total dosage will be between 10 μg and 1 ng. When the nucleic acid construct is administered directly, the total dosage is generally higher, for example between 50 μg and 1 or more milligram. The above dosages are exemplary of the average case.

In a protein vaccine, the amount of protein in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, most preferably 1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be either a priming or boosting vaccination regime; be administered systemically, for example via the transdermal, subcutaneous or intramuscular routes or applied to a mucosal surface via, for example, intra nasal or oral routes.

There can, of course, be individual instances where higher or lower dosage ranges are merited, and such are within the scope of this invention.

It is possible for the vaccine composition to be administered on a once off basis or to be administered repeatedly, for example, between 1 and 7 times, preferably between 1 and 4 times, at intervals between about 1 day and about 18 months, preferably one month. This may be optionally followed by dosing at regular intervals of between 1 and 12 months for a period up to the remainder of the patient's life. In an embodiment the patient will receive the antigen in different forms in a prime boost regime. Thus for example an antigen will be first administered as a DNA based vaccine and then subsequently administered as a protein adjuvant base formulation. Once again, however, this treatment regime will be significantly varied depending upon the size and species of animal concerned, the amount of nucleic acid vaccine and/or protein composition administered, the route of administration, the potency and dose of any adjuvant compounds used and other factors which would be apparent to a skilled veterinary or medical practitioner.

Throughout this specification the words “comprise” and “include” or variations such as “comprising”, “comprises”, “including”, “includes” etc., are to be construed both inclusively, that is, use of these words will imply the possible inclusion of integers or elements not specifically recited and also in the exclusionary sense in that the words could be read as “consisting”.

As described herein, the present invention relates isolated polypeptides and isolated polynucleotides. In the context of this invention the term “isolated” is intended to convey that the polypeptide or polynucleotide is not in its native state, insofar as it has been purified at least to some extent or has been synthetically produced, for example by recombinant methods, or mechanical synthesis. The term “isolated” therefore includes the possibility of the polypeptides or polynucleotides being in combination with other biological or non-biological material, such as cells, suspensions of cells or cell fragments, proteins, peptides, expression vectors, organic or inorganic solvents, or other materials where appropriate, but excludes the situation where the polynucleotide is in a state as found in nature.

The present invention is exemplified, but not limited to, the following examples.

### EXAMPLES

1. Design of a Vaccine Against Murine IL-13

IL-13 belongs to the SCOP (Murzin et al, 1995, J Mol Biol 247:536-540) defined 4 helical cytokines fold family. Individual members of this fold superfamily are related structurally, but are difficult to align at the sequence level. The 3D structure of IL-13 has not yet been determined, but structures have been generated for a number of other 4-helical cytokines. Protein multiple sequence alignments were generated for IL-13 orthologues, and also for a number of other cytokines exhibiting this fold where the structure of at least one member had been determined (IL-4, GM-CSF, IL-5 and IL-2). Secondary structure predictions were performed for the IL-13 protein multiple sequence alignment using DSC (King and Stemberg, 1996, Prot Sci 5:2298-2310), SIMPA96 (Levin, 1997, Prot Eng 7:771-776) and Pred2ary (Chandonia and Karplus, 1995, Prot Sci 4:275-285). The individual cytokine protein multiple sequence alignments were aligned to each other, using both the sequence information and the structural information (from the known crystal structures and from the secondary structure prediction).

Antigenic sites, specifically B-cell epitopes, were predicted for murine IL-13 using the Cameleon software (Oxford Molecular), and these were mapped onto the IL-4 structure (accession number 1RCB in the Brookhaven database) using the protein multiple sequence alignment to give an idea of where they might be located structurally on IL-13. From this analysis, exposed regions which were potentially both antigenic and involved in receptor binding were selected.

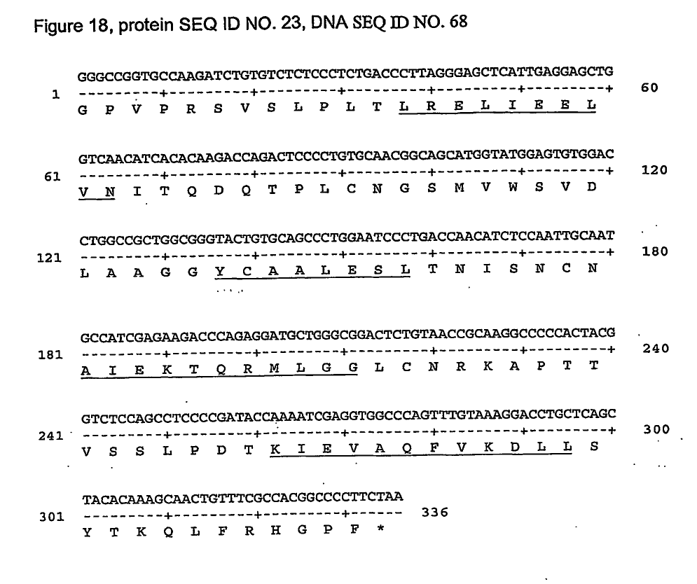

From this model, a chimaeric IL-13 sequence was designed in which the sequence of the predicted antigenic loops was taken from murine IL-13, and the sequence of the predicted structural (predominantly helical) regions was taken from human IL-13. The purpose of this design was to identify target epitopes from murine IL-13 against which neutralising antibodies might be raised, and to present them on a framework which was structurally similar to the native protein, but yet contained sufficient sequence variation to the native (murine) protein to ensure that one or more CD4 T helper epitopes would be present. The nucleic acid and protein sequences selected for this example of a chimaeric IL-13 vaccine are shown in FIG. 18 (SEQ ID NO 23). The underlined sequences correspond to sequences found in the human orthologue. Twelve amino acids were substituted to achieve the sequence in FIG. 18. It should be understood that the degeneracy of the genetic code allows many possible nucleic acid sequences to encode identical proteins. Furthermore, it will be appreciated that there are other possible chimaeric IL-13 vaccine designs within the scope of the invention, that have other orthologus mutations in non-exposed areas.

1.2 Preparation of Chimaeric IL-13

Chimaeric IL-13 (cIL-13) DNA sequence was synthesised from a series of partially overlapping DNA oligonucleotides, with the sequences cIL-13-1 to cIL-13-6 shown in Table 1. These oligos were annealed, and cIL-13 DNA generated by a PCR with the cycle specification of 94° C. for 1 minute followed by 25 cycles of 94° C. for 30 seconds, 55° C. for 1 minute and 72° C. for 2 minutes. Followed by 72° C. for 7 minutes and cooling to 4° C. when finished. The reaction product comprised a band of the expected size, 361 base pairs, which was subcloned into the T/A cloning vector pCR2.1 (Invitrogen, Groningen, Netherlands) to generate pCR2.1-cIL 13. A BamH1 and Xho1 cIL-13 digested fragment from pCR2.1-cIL-13 was then subcloned into the BamH1 and Xho1 sites in pGEX4T3 (Amersham Pharmacia, Amersham, Bucks, UK) generating pGEX4T3-cIL-13/1. On sequencing the pGEX4T3-cIL-13/1 construct we discovered an extra 39 base pairs of DNA sequence (derived from the pCR2.1 vector) between the sequence for GST and cIL-13. To correct this, we repeated the PCR for cIL-13 using pGEX4T3-cIL-13/1 and primers cIL-13Fnew and cIL-13R. The PCR product obtained was then cloned back into pGEX4T3 using BamH1 and Xho1 restriction sites, to generate the expression vector pGEX4T3-cIL-13. The sequence of this construct was verified by dideoxy terminator sequencing. This vector encodes a genetic fusion protein consisting of glutathione-S-transferase and cIL-13 (GST-cIL 13). The two moieties of the protein are linked by a short spacer which contains the recognition site for thrombin. The fusion protein may be readily purified by glutathione sepharose affinity chromatography, and then used directly, or a preparation of free cIL-13 produced by cleavage with thrombin.

TABLE 1
Oligonucleotides used to construct chimaeric IL-13.

| | |
|---|---|
| Oligo | Sequence (5′-3′) |

| | | |
|---|---|---|
| cIL-13-1R | (SEQ ID NO 49) | TGTGATGTTGACCAGCTCCTCAATGAGCTCCCTAAGGGT |
| | | CAGAGGGAGAGACACAGATCTTGGCACCGGCCC |
| cIL-13-2F | (SEQ ID NO 50) | AGGAGCTGGTCAACATCACACAAGACCAGACTCCCCTG |
| | | TGCAACGGCAGCATGGTATGGAGTGTGGACCTGGC |
| cIL-13-3R | (SEQ ID NO 51) | GCAATTGGAGATGTTGGTCAGGGATTCCAGGGCTGCAC |
| | | AGTACCCGCCAGCGGCCAGGTCCACACTCCATAC |
| cIL-13-4F | (SEQ ID NO 52) | TGACCAACATCTCCAATTGCAATGCCATCGAGAAGACC |
| | | CAGAGGATGCTGGGCGGACTCTGTAACCGCAAGGC |
| cIL-13-5R | (SEQ ID NO 53) | AAACTGGGCCACCTCGATTTTGGTATCGGGGAGGCTGG |
| | | AGACCGTAGTGGGGGCCTTGCGGTTACAGAGTCC |
| cIL-13-6F | (SEQ ID NO 54) | AAATCGAGGTGGCCCAGTTTGTAAAGGACCTGCTCAGC |
| | | TACACAAAGCAACTGTTTCGCCACGGCCCCTTC |
| cIL-13F | (SEQ ID NO 55) | CGCGGATTCGGGCCGGTGCCAAGATCTG |
| cIL-13R | (SEQ ID NO 56) | CTCCGCTCGAGTCGACTTAGAAGGGGCCGTGGCGAAA |
| cIL-13Fnew | (SEQ ID NO 57) | CGCGGATCCGGGCCGGTGCCAAGATCTG |

The pGEX4T3-cIL-13 expression vector was transformed into E. coli BLR strain (Novagen, supplied by Cambridge Bioscience, Cambridge, UK). Expression of GST-cIL-13 was induced by adding 0.5 mM IPTG to a culture in the logarithmic growth phase for 4 hrs at 37° C. The bacteria were then harvested by centrifugation and GST-cIL-13 purified from them by a method previously described for purification of a similar GST-human IL-13 fusion protein (McKenzie et al, 1993, Proc Natn Acad Sci 90:3735-3739).
2. In vitro Mouse IL-13 Neutralisation Bioassay.
To measure the ability of vaccine generated IL-13 antiserum to neutralise the bioactivity of recombinant mouse IL-13 on human TF-1 cells (obtained in-house), 5 ng/ml recombinant mouse IL-13 was incubated with various concentrations of sera for 1 hour at 37° C. in a 96-well tissue culture plate (Invitrogen). Following this pre-incubation period, TF-1 cells were added. The assay mixture, containing various serum dilutions, recombinant mouse IL-13 and TF-1 cells, was incubated at 37° C. for 70 hours in a humidified CO₂ incubator. MTT substrate (Cat. No. G4000, Promega) was added during the final 4 hours of incubation, after which the reaction was stopped with an acid solution to solubilise the metabolised blue formazan product. The absorbance of the solution in each well was read in a 96-well plate reader at 570 nm wavelength.
Note that this assay is only able to measure mouse IL-13 neutralisation capacity in serum dilutions greater than or equivalent to 1/100. Serum dilutions less than 1/100 induce non-specific proliferative effects in TF-1 cells.
The capacity of the serum to neutralise mouse IL-13 bioactivity was expressed as, that dilution of serum required to neutralise the bioactivity of a defined amount of mouse IL-13 by 50% (=ND₅₀). The more dilute serum sample required, the more potent the neutralisation capacity.
2.5 Determination of the Level of Mouse IL-13 Neutralisation Required for Efficacy in the ‘Ovalbumin Challenge’ Mouse Asthma Model.

In order to benchmark the required potency of an IL-13 autovaccine for treatment of asthma, mice were treated with various doses of rabbit anti-mouse IL-13 polyclonal antibody (administered passively by intra-peritoneal injection) during ovalbumin challenge, in the ‘ovalbumin challenge’ mouse asthma model. Model parameters such as airway hyper-responsiveness (AHR), goblet cell metaplasia (GCM) and lung inflammatory cell content were measured at the end of this experiment. Efficacy in this model was correlated to the levels of mouse IL-13 neutralisation achieved in mouse serum. The mouse IL-13 neutralisation bioassay was used to determine the level of mouse IL-13 neutralisation in serum samples.

| Treatment group | |
|---|---|
| (Dose of passively | Mouse IL-13 |
| administered rabbit anti- | neutralisation capacity |
| mouse IL-13 antibody) | (ND₅₀) |
| Highest dose | 1/4100 |
| High dose | 1/2670 |
| Mid dose | 1/476 |
| Lowest dose | 1/207 |

Treatment groups given the highest three doses of antibody all performed similarly. All of these three groups showed efficacy equivalent to (for AHR) or better than (for GCM) the gold standard treatment (dexamethasone, administered by the intraperitoneal route at 3×1.5 mg/kg) used in this model. The ‘lowest dose’ of antibody administered, showed efficacy somewhere between that of dexamethasone and the ‘no treatment’ positive control groups.
Therefore the level of IL-13 neutralisation achieved in the ‘mid dose’ treatment group, represents the required potency threshold for an IL-13 autovaccine in this animal model. The potency threshold is defined as the lowest level of IL-13 neutralisation in mouse serum, required to show 100% efficacy in the asthma model (=ED₁₀₀). 1×ED₁₀₀ is therefore equivalent to an ND₅₀ of 1/476.
3. Vaccination Studies
Mice are immunised with protein in adjuvant. The primary immunisation will use ˜100 ug protein, followed by ˜50 ug for subsequent boost immunisations. Immunisations will be administered on a 4 weekly basis, serum samples will be taken from the mice 2 weeks after each immunisation (in order to monitor the level of anti-mouse IL13 antibodies and the IL13 neutralisation capacity generated in these serum samples).

1. A vaccine composition for the treatment of asthma, comprising an immunogen that generates an immune response in a vaccinee against self IL-13 and an adjuvant comprising a combination of an immunostimulatory oligonucleotide containing at least one unmethylated CG motif and a saponin.

2. The vaccine as claimed in claim 1 wherein the immunogen generates an immune response against human IL13.

3. The vaccine as claimed in claim 2 wherein the immunogen comprises human IL-13 supplemented with foreign T-helper epitopes.

4. The vaccine as claimed in claim 2, wherein the immunogen comprises a non-human IL-13 backbone, substituted with human IL-13 B cell epitopes.

5. The vaccine as claimed in claim 1 wherein the saponin is QS21.

6. The vaccine as claimed in claim 1 wherein the immunostimulatory oligonucleotide has the sequence TCG TCG TTT TGT CGT TTT GTC GTT (SE ID NO. 47).

7. The vaccine as claimed in claim 1 wherein the vaccine comprises a human IL-13 immunogen comprising an orthologous IL-13 sequence, wherein at least one of the orthologous B-cell epitopes is substituted for the equivalent human sequences.

8. A vaccine composition for the treatment of COPD, comprising an immunogen that generates an immune response in a vaccinee against self IL-13 and an adjuvant comprising a combination of an immunostimulatory oligonucleotide containing at least one unmethylated CG motif and a saponin.


SEQUENCE LISTING (68 sequences)

SEQ ID NO: 1 - protein, 112 aa, Homo Sapien IL-13
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 2 - protein, 111 aa, Murine IL-13
GPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 3 - protein, 111 aa, Porcine IL-13
GPVPPHSTALKELIEELVNITQNQKTPLCNGSMVWSVNLTTSMQYCAALESLINISDCSAIQKTQRMLSALCSHKPPSEQVPGKHIRDTKIEVAQFVKDLLKHLRMIFRHG

SEQ ID NO: 4 - protein, 112 aa, Bovine IL-13
SPVPSATALKELIEELVNITQNQKVPLCNGSMVWSLNLTSSMYCAALDSLISISNCSVIQRTKKMLNALCPHKPSAKQVSSEYVRDTKIEVAQFLKDLLRHSRIVFRNERFN

SEQ ID NO: 5 - protein, 111 aa, Canine IL-13
SPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 6 - protein, 111 aa, Rat IL-13
GPVRRSTSPPVALRELIEELSNITQDQKTSLCNSSMVWSVDLTAGGFCAALESLTNISSCNAIHRTQRILNGLCNQKASDVASSPPDTKIEVAQFISKLLNYSKQLFRYGH

SEQ ID NO: 7 - protein, 111 aa, Cynomolgus IL-13
SPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLHLKKLFREGQFN

SEQ ID NO: 8 - protein, 112 aa, Rhesus IL-13
SPVPRSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGRFN

SEQ ID NO: 9 - protein, 112 aa, Marmoset IL-13
GPVPPYTALKELIEELVNITQNQKAPLCNGSMVWSINMTAGVYCAALESLINVSGCSAIEKTQRMLSGFCPHKVSAGQFSSLLVRDTKIEVAQFVKDLLRHLRKLFHQGTFN

SEQ ID NO: 10 - protein, 112 aa, Artificial Sequence
  Chimaeric Homo Sapien IL-13
GPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 11 - protein, 121 aa, Artificial Sequence
  Chimaeric Homo Sapien IL-13
GPVPPSTALRELIEELVNITQNQKAPLCNGSMVWSINLTAGMYCAALESLINVSGCSAIEKTQRMLGGFCPHKFNNFTVSFWLRVPKVSASHLEDTKIEVAQFVKDLLLHLKKLFREGRFN

SEQ ID NO: 12 - protein, 133 aa, Artificial Sequence
  Chimaeric Homo Sapien IL-13
FNNFTVSFWLRVPKVSASHLEGPVPPSTALRELIEELVNITQNQKAPLCNGSMVWSINLTAGMYCAALESLINVSGCSAIEKTQRMLGGFCPHKVSAGQFSSLHVRDTKIEVAQFVKDLLLHLKKLFREGRFN

SEQ ID NO: 13 - protein, 123 aa, Artificial Sequence
  Chimaeric Murine IL-13
GPVPRSVSLPLTLKELIEELSNITQDQTPLCNGSMVWSVDLAAGGFCVALDSLTNISNCNAIYRTQRILHGLCNRKFNNFTVSFWLRVPKVSASHLEDTKIEVAHFITKLLSYTKQLFRHGPF

SEQ ID NO: 14 - protein, 132 aa, Artificial Sequence
  Chimaeric Murine IL-13
FNNFTVSFWLRVPKVSASHLEGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 15 - protein, 132 aa, Artificial Sequence
  Chimaeric Murine IL-13
FNNFTVSFWLRVPKVSASHLEGPVPRSVSLPVTLKELIEELTNITQDQTPLCNGSMVWSVDLAAGGFCVALDSLTNISNCNAIFRTQRILHALCNRKAPTTVSSLPDTKIEVAHFITKLLTYTKNLFRRGPF

SEQ ID NO: 16 - protein, 249 aa, Artificial Sequence
  Chimaeric Homo Sapien IL-13
YVHSDGSYPKDKFEKINGTWYYFDSSGYMLADRWRKHTDGNWYWFDNSGEMATGWKKIADKWYYFNEEGAMKTGWVKYKDTWYYLDAKEGAMQYIKANSKFIGITEGVMVSNAFIQSADGTGWYYLKPDGTLADRPEGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 17 - protein, 220 aa, Artificial Sequence
  Chimaeric Homo Sapien IL-13
SSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 18 - protein, 133 aa, Artificial Sequence
  Chimaeric Homo Sapien IL-13
FNNFTVSFWLRVPKVSASHLEGPVPPSSALKELIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 19 - protein, 133 aa, Artificial Sequence
  Chimaeric Homo Sapien IL-13
FNNFTVSFWLRVPKVSASHLEGPVPPSSALKILIEELANITQNQKAPLCNGSMVWSINLTAGMYCAALDSLINVSGCSAIERTQRILSAFCPHKVSAGQFSSLRVRDTKIEVAQFVTDLLVHLKRLFRQGTFN

SEQ ID NO: 20 - protein, 112 aa, Artificial Sequence
  Chimaeric Homo Sapien IL-13
GPVPPSSALKELIEELANITQNQKAPLCNGSMVWSINLTAGMYCAALDSLINVSGCSAIERTQRILSAFCPHKVSAGQFSSLHVRDTKIEVAQFVTDLLVHLKRLFRQGRFN

SEQ ID NO: 21 - protein, 112 aa, Artificial Sequence
  Chimaeric Homo Sapien IL-13
GPVPPSTALKELIEELVNITQNQKAPLCNGSMVWSINLTAGMYCAALDSLINVSGCSAIERTQRILSAFCPHKVSAGQFSSLRVRDTKIEVAQFVTDLLVHLKKLFRQGTFN

SEQ ID NO: 22 - protein, 112 aa, Artificial Sequence
  Chimaeric Homo Sapien IL-13
GPVPPSSALRELIEELANITQNQKAPLCNGSMVWSINLTAGMYCAALESLINVSGCSAIDKTQRMLSAFCPHKVSAGQFSSLHVRDTKIEVAQFVKDLLVHLKRLFRDGRFN

SEQ ID NO: 23 - protein, 111 aa, Artificial Sequence
  Chimaeric Homo Sapien IL-13
GPVPRSVSLPLTLRELIEELVNITQDQTPLCNGSMVWSVDLAAGGYCAALESLTNISNCNAIEKTQRMLGGLCNRKAPTTVSSLPDTKIEVAQFVKDLLSYTKQLFRHGPF

SEQ ID NO: 24 - protein, 16 aa, Homo Sapien
PSTALRELIEELVNIT

SEQ ID NO: 25 - protein, 10 aa, Homo Sapien
MYCAALESLI

SEQ ID NO: 26 - protein, 9 aa, Homo Sapien
KTQRMLSGF

SEQ ID NO: 27 - protein, 17 aa, Homo Sapien
AQFVKDLLLHLKKLFRE

SEQ ID NO: 28 - protein, 8 aa, Homo Sapien
GPVPPSTA

SEQ ID NO: 29 - protein, 24 aa, Homo Sapien
ITQNQKAPLCNGSMVWSINLTAGM

SEQ ID NO: 30 - protein, 7 aa, Homo Sapien
INVSGCS

SEQ ID NO: 31 - protein, 19 aa, Homo Sapien
FCPHKVSAGQFSSLHVRDT

SEQ ID NO: 32 - protein, 13 aa, Homo Sapien
LHLKKLFREGRFN

SEQ ID NO: 33 - protein, 14 aa, Clostridium tetani
QYIKANSKFIGITE

SEQ ID NO: 34 - protein, 21 aa, Clostridium tetani
FNNFTVSFWLRVPKVSASHLE

SEQ ID NO: 35 - protein, 21 aa, Plasmodium falciparum
DIEKKIAKMEKASSVFNVVNS

SEQ ID NO: 36 - protein, 15 aa, Paramyxoviridae Morbillivirus
LSEIKGVIVHRLEGV

SEQ ID NO: 37 - protein, 15 aa, Hepatitis B virus
FFLLTRILTIPQSLD

SEQ ID NO: 38 - protein, 19 aa, Corynebacterium diphtheriae
PVFAGANYAAWAVNVAQVI

SEQ ID NO: 39 - protein, 20 aa, Corynebacterium diphtheriae
VHHNTEEIVAQSIALSSLMV

SEQ ID NO: 40 - protein, 20 aa, Corynebacterium diphtheriae
QSIALSSLMVAQAIPLVGEL

SEQ ID NO: 41 - protein, 20 aa, Corynebacterium diphtheriae
VDIGFAAYNFVESIINLFQV

SEQ ID NO: 42 - protein, 20 aa, Corynebacterium diphtheriae
QGESGHDIKITAENTPLPIA

SEQ ID NO: 43 - protein, 20 aa, Corynebacterium diphtheriae
GVLLPTIPGKLDVNKSKTHI

SEQ ID NO: 44 - DNA, 20 bp, Artificial Sequence
  artificial immunostimulatory oligonucleotide
tccatgacgttcctgacgtt

SEQ ID NO: 45 - DNA, 18 bp, Artificial Sequence
  artificial immunostimulatory oligonucleotide
tctcccagcgtgcgccat

SEQ ID NO: 46 - DNA, 30 bp, Artificial Sequence
  artificial immunostimulatory oligonucleotide
accgatgacgtcgccggtgacggcaccacg

SEQ ID NO: 47 - DNA, 24 bp, Artificial Sequence
  artificial immunostimulatory oligonucleotide
tcgtcgttttgtcgttttgtcgtt

SEQ ID NO: 48 - DNA, 20 bp, Artificial Sequence
  artificial immunostimulatory oligonucleotide
tccatgacgttcctgatgct

SEQ ID NO: 49 - DNA, 72 bp, Artificial Sequence
  primer
tgtgatgttgaccagctcctcaatgagctccctaagggtcagagggagagacacagatcttggcaccggccc

SEQ ID NO: 50 - DNA, 73 bp, Artificial Sequence
  primer
aggagctggtcaacatcacacaagaccagactcccctgtgcaacggcagcatggtatggagtgtggacctggc

SEQ ID NO: 51 - DNA, 72 bp, Artificial Sequence
  primer
gcaattggagatgttggtcagggattccagggctgcacagtacccgccagcggccaggtccacactccatac

SEQ ID NO: 52 - DNA, 73 bp, Artificial Sequence
  primer
tgaccaacatctccaattgcaatgccatcgagaagacccagaggatgctgggcggactctgtaaccgcaaggc

SEQ ID NO: 53 - DNA, 72 bp, Artificial Sequence
  primer
aaactgggccacctcgattttggtatcggggaggctggagaccgtagtgggggccttgcggttacagagtcc

SEQ ID NO: 54 - DNA, 71 bp, Artificial Sequence
  primer
aaatcgaggtggcccagtttgtaaaggacctgctcagctacacaaagcaactgtttcgccacggccccttc

SEQ ID NO: 55 - DNA, 28 bp, Artificial Sequence
  primer
cgcggattcgggccggtgccaagatctg

SEQ ID NO: 56 - DNA, 37 bp, Artificial Sequence
  primer
ctccgctcgagtcgacttagaaggggccgtggcgaaa

SEQ ID NO: 57 - DNA, 28 bp, Artificial Sequence
  primer
cgcggatccgggccggtgccaagatctg

SEQ ID NO: 58 - protein, 6 aa, Homo Sapien IL-13
ELIEEL

SEQ ID NO: 59 - protein, 4 aa, Homo Sapien IL-13
NITQ

SEQ ID NO: 60 - protein, 5 aa, Homo Sapien IL-13
SMVWS

SEQ ID NO: 61 - protein, 7 aa, Homo Sapien IL-13
DTKIEVA

SEQ ID NO: 62 - DNA, 336 bp, Chimeric Homo Sapien IL-13
ggccctgtgcctccctctagcgccctcaaggagctcattgaggagctggccaacatcacccagaaccagaaggctccgctctgcaatggcagcatggtatggagcatcaacctgacagctggcatgtactgtgcagccctggactccctgatcaacgtgtcaggctgcagtgccatcgagcggacccagaggatcttgagcgccttctgcccgcacaaggtctcagctgggcagttttccagcttgcgtgtccgagacaccaaaatcgaggtggcccagtttgtaacggacctgctcgtacatttaaagagactttttcgccagggaacgttcaac

SEQ ID NO: 63 - DNA, 336 bp, Chimeric Homo Sapien IL-13
ccgggacacggagggagatcgcgggagttcctcgagtaactcctcgaccggttgtagtgggtcttggtcttccgaggcgagacgttaccgtcgtaccatacctcgtagttggactgtcgaccgtacatgacacgtcgggacctgagggactagttgcacagtccgacgtcacggtagctcgcctgggtctcctagaactcgcggaagacgggcgtgttccagagtcgacccgtcaaaaggtcgaacgcacaggctctgtggttttagctccaccgggtcaaacattgcctggacgagcatgtaaatttctctgaaaaagcggtcccttgcaagttg

SEQ ID NO: 64 - DNA, 747 bp, Chimeric Homo Sapien IL-13
tacgtacattccgacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatgcaatacatcaaggctaactctaagttcattggtatcactgaaggcgtcatggtatcaaatgcctttatccagtcagcggacggaacaggctggtactacctcaaaccagacggaacactggcagacaggccagaaggccctgtgcctccctctagcgccctcaaggagctcattgaggagctggccaacatcacccagaaccagaaggctccgctctgcaatggcagcatggtatggagcatcaacctgacagctggcatgtactgtgcagccctggactccctgatcaacgtgtcaggctgcagtgccatcgagcggacccagaggatcttgagcgccttctgcccgcacaaggtctcagctgggcagttttccagcttgcgtgtccgagacaccaaaatcgaggtggcccagtttgtaacggacctgctcgtacatttaaagagactttttcgccagggaacgttcaac

SEQ ID NO: 65 - DNA, 660 bp, Chimeric Homo Sapien IL-13
tcctctcattcttctaacatggcgaacacccagatgaagtccgataaaatcatcatcgcgcacaggggagctagcgggtatctgcctgagcacaccctggagtccaaggctctggcgttcgcccagcaggctgactacctggagcaggacctggcgatgacaaaggatggccgcctcgtggtgatccatgaccattttctcgacggactgaccgacgtcgccaagaagttcccccaccgccataggaaggacgggaggtattacgtgattgacttcaccctcaaggagatccagagcctggagatgaccgagaacttcgagaccggccctgtgcctccctctagcgccctcaaggagctcattgaggagctggccaacatcacccagaaccagaaggctccgctctgcaatggcagcatggtatggagcatcaacctgacagctggcatgtactgtgcagccctggactccctgatcaacgtgtcaggctgcagtgccatcgagcggacccagaggatcttgagcgccttctgcccgcacaaggtctcagctgggcagttttccagcttgcgtgtccgagacaccaaaatcgaggtggcccagtttgtaacggacctgctcgtacatttaaagagactttttcgccagggaacgttcaac

SEQ ID NO: 66 - DNA, 399 bp, Chimeric Homo Sapien IL-13
tttaataattttaccgttagcttttggttgcgtgttcctaaagtatctgctagtcatttagaaggccctgtgcctccctctagcgccctcaaggagctcattgaggagctggccaacatcacccagaaccagaaggctccgctctgcaatggcagcatggtatggagcatcaacctgacagctggcatgtactgtgcagccctggactccctgatcaacgtgtcaggctgcagtgccatcgagcggacccagaggatcttgagcgccttctgcccgcacaaggtctcagctgggcagttttccagcttgcgtgtccgagacaccaaaatcgaggtggcccagtttgtaacggacctgctcgtacatttaaagagactttttcgccagggaacgttcaac

SEQ ID NO: 67 - DNA, 399 bp, Chimeric Homo Sapien IL-13
tttaataattttaccgttagcttttggttgcgtgttcctaaagtatctgctagtcatttagaaggccctgtgcctccctctagcgccctcaagattctcattgaggagctggccaacatcacccagaaccagaaggctccgctctgcaatggcagcatggtatggagcatcaacctgacagctggcatgtactgtgcagccctggactccctgatcaacgtgtcaggctgcagtgccatcgagcggacccagaggatcttgagcgccttctgcccgcacaaggtctcagctgggcagttttccagcttgcgtgtccgagacaccaaaatcgaggtggcccagtttgtaacggacctgctcgtacatttaaagagactttttcgccagggaacgttcaac

SEQ ID NO: 68 - DNA, 336 bp, Chimeric Homo Sapien IL-13
gggccggtgccaagatctgtgtctctccctctgacccttagggagctcattgaggagctggtcaacatcacacaagaccagactcccctgtgcaacggcagcatggtatggagtgtggacctggccgctggcgggtactgtgcagccctggaatccctgaccaacatctccaattgcaatgccatcgagaagacccagaggatgctgggcggactctgtaaccgcaaggcccccactacggtctccagcctccccgataccaaaatcgaggtggcccagtttgtaaaggacctgctcagctacacaaagcaactgtttcgccacggccccttctaa